(19) United States
(12) Patent Application Publication
(10) Pub. No.: US 2004/0220252 A1
(43) Pub. Date: Nov. 4, 2004

(54) Novel crystalline forms of trandolapril

(76) Inventors: Reddy Bandi Parthasaradhi, Hyderabad (IN); Reddy Kura Rathnakar, Hyderabad (IN); Reddy Rapolu Raji, Hyderabad (IN); Reddy Bolla Narasa, Hyderabad (IN); Reddy Dasari Muralidhara, Hyderabad (IN)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(21) Appl. No.: 10/250,654

(22) PCT Filed: Feb. 27, 2003
(86) PCT No.: PCT/IN03/00038

(51) Int. Cl. A61K 31/405; C07D 209/42

(52) U.S. Cl. 514/419; 548/492

(57) ABSTRACT

The present invention relates to two novel crystalline polymorphs of trandolapril, processes for their preparation and the pharmaceutical compositions containing them.

### FIELD OF THE INVENTION

The present invention relates to two novel crystalline polymorphs of trandolapril, processes for their preparation and the pharmaceutical compositions containing them.

### BACKGROUND OF THE INVENTION

(2S,3aR,7aS)-1-[(2S)-2-[[(1S)-1-(Ethoxycarbonyl)-3-phenylpropyl]amino]-1-oxopropyl]octahydro-1H-indole-2-carboxylic acid (Trandolapril) is an angiotensin converting enzyme inhibitor, which was described along with related compounds in U.S. Pat. No. 4,933,361. The process for the synthesis of trandolapril was described in U.S. Pat. No. 4,933,361 and WO 96/33984. No crystalline form was referred and no process for the crystallization of trandolapril was described in U.S. Pat. No. 4,933,361 and WO 96/33984. It has now been discovered that trandolapril can exist in two different polymorphic crystalline forms, which differ from each other by their spectral characteristics and the process for their preparation.

According to U.S. Pat. No. 4,933,361, benzyl N-(1S-carboethoxy-3-phenyl propyl)-S-alanyl-2S,3aR,7aS-octahydroindole-2-carboxylate was hydrogenated under normal pressure in ethanol in the presence of 10% palladium charcoal and the solvent evaporated to give poorly crystalline trandolapril foam.

Thus there is a need to prepare trandolapril in stable and consistently reproducible crystalline forms.

The present invention relates to two novel crystalline polymorphs of trandolapril, which are useful as a medicine as described in U.S. Pat. No. 4,933,361. These new crystalline forms of trandolapril are useful as active ingredient for the preparation of a medicine.

Thus, one object of the present invention is to provide two novel crystalline polymorphic forms of trandolapril and process for their preparation.

Another object of the present invention is to provide a pharmaceutical formulation comprising either or both of the novel crystalline polymorphs of trandolapril and a pharmaceutically acceptable carrier.

### BRIEF DESCRIPTION OF THE DRAWINGS

FIG. 1 is a powder x-ray diffractogram of Form I crystalline polymorph of trandolapril.

FIG. 2 is a powder x-ray diffractogram of Form II crystalline polymorph of trandolapril.

Powder x-ray diffraction pattern was measured on a Siemens D-5000 diffractometer.

### DESCRIPTION OF THE INVENTION

In accordance with one feature of the present invention there is provided a novel crystalline polymorph of (2S,3aR,7aS)-1-[(2S)-2-[[(1S)-1-(Ethoxycarbonyl)-3-phenylpropyl]amino]-1-oxopropyl]octahydro-1H-indole-2-carboxylic acid (Trandolapril) designated as Form I crystalline polymorph of trandolapril having Characteristic peaks expressed as 20 values at approximately 7.7, 9.0, 11.0, 12.6, 14.9, 15.5, 15.9, 17.0, 17.4, 17.7, 18.8, 19.9, 20.5, 23.2, 24.3, 29.0 degree.

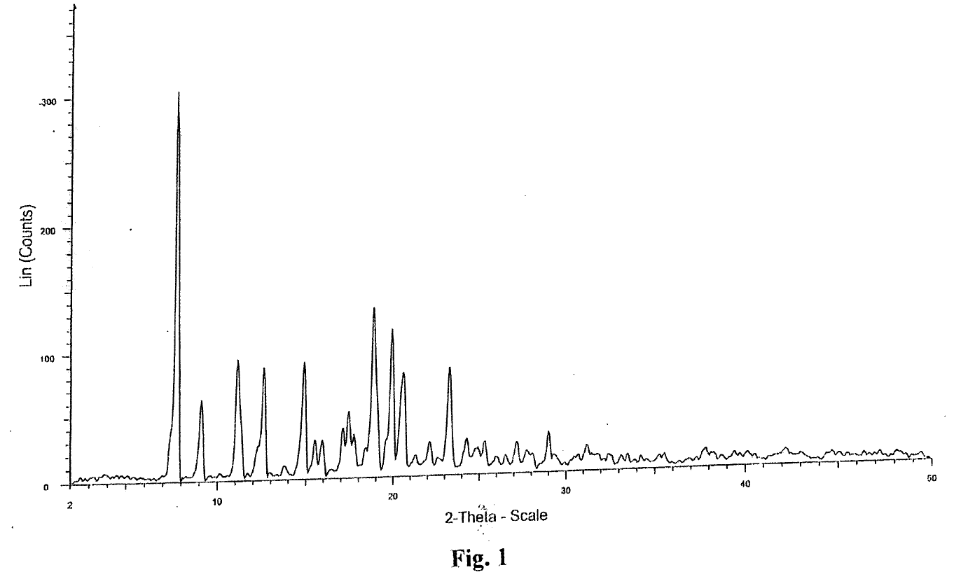

x-Ray powder diffraction spectra of Form I is depicted in FIG. 1. The significant reflections of Form I are shown in table 1. The intensities are expressed as percentage of most intense peak.

In accordance with an another feature of the present invention, there is provided a process for the preparation of Form I crystalline polymorph of trandolapril. Form I crystalline polymorph of trandolapril is prepared by mixing trandolapril obtained in U.S. Pat. No. 4,933,361 or Form II crystalline polymorph of trandolapril obtained by the process described below and diisopropyl ether; refluxing for about 15 to about 45 minutes; cooling to about 15 to about 35° C.; and maintaining the solution at about 15 to about 35° C. for about 15 minutes to about 2 hours. The reflux time in this process is preferably for about 30 minutes and the maintenance is preferably for about 1 hour at about 20 to about 25° C. During maintenance contents may be seeded with Form I crystalline polymorph of trandolapril.

In accordance with another feature of the present invention there is provided a novel crystalline polymorph of (2S,3aR,7aS)-1-[(2S)-2-[[(1S)-1-(Ethoxycarbonyl)-3-phenylpropyl]amino]-1-oxopropyl]octahydro-1H-indole-2-carboxylic acid (Trandolapril) designated as Form II crystalline polymorph of trandolapril having characteristic peaks expressed as 20 values at approximately 7.3, 8.9, 12.2, 12.5, 14.6, 17.0, 17.8, 18.7, 19.8, 21.5, 22.1, 25.2, 27.8, 29.6 degree.

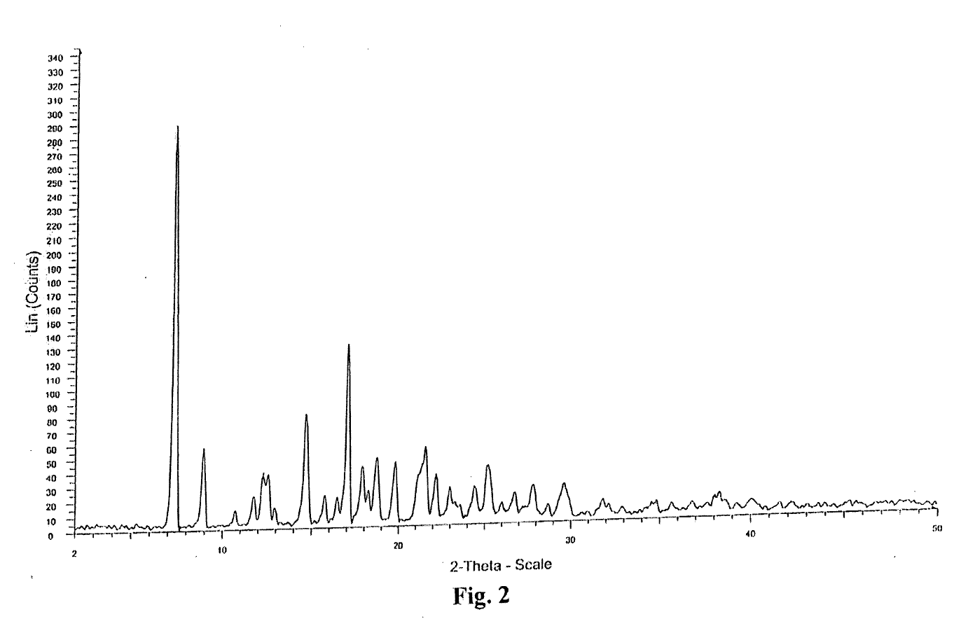

x-Ray powder diffraction spectra of Form II is depicted in FIG. 2. The significant reflections of Form II are shown in table 2. The intensities are expressed as percentage of most intense peak.

In accordance with an another feature of the present invention, there is provided a process for the preparation of Form II crystalline polymorph of trandolapril. Form II crystalline polymorph of trandolapril is prepared by mixing trandolapril obtained by the process described in U.S. Pat. No. 4,933,361 or Form II crystalline polymorph of trandolapril obtained by the process described above and ethylacetate; refluxing for about 30 minutes; cooling to about 15 to 35° C.; and maintaining the solution at about 15-35° C. for about 15 minutes to 3 hours. The reflux time in this process is preferably for about 30 minutes and the maintenance is preferably for about 30 minutes at about 20 to about 25° C. During maintenance contents may be seeded with Form II crystalline polymorph of trandolapril.

In accordance with an another feature of the present invention, there is provided a pharmaceutical composition comprising either Form I or Form II or mixture thereof of trandolapril and a pharmaceutically acceptable carrier. Suitable pharmaceutical carriers include solid diluents or filters, various organic solvents and excipients known to those skilled in the art.

The present invention is illustrated by the following examples, but it is not limited to the details thereof.

TABLE 1

| 2θ (degree) | % Intensity |
|---|---|
| 7.7 | 100 |
| 9.0 | 20.8 |
| 11.0 | 31.5 |
| 12.6 | 29.4 |
| 14.9 | 30.7 |
| 15.5 | 9.6 |
| 15.9 | 9.9 |
| 17.0 | 12.2 |
| 17.4 | 16.9 |
| 17.7 | 10.7 |
| 18.8 | 44.5 |
| 19.9 | 38.8 |
| 20.5 | 28.7 |
| 23.2 | 28.3 |
| 24.3 | 9.2 |
| 29.0 | 9.9 |

TABLE 2

| 2θ (degree) | % Intensity |
|---|---|
| 7.3 | 100.0 |
| 8.9 | 19.0 |
| 12.2 | 12.8 |
| 12.5 | 12.9 |
| 14.6 | 27.7 |
| 17.0 | 44.2 |
| 17.8 | 14.4 |
| 18.7 | 16.8 |
| 19.8 | 15.6 |
| 21.5 | 18.8 |
| 22.1 | 11.7 |
| 25.2 | 14.3 |
| 27.8 | 9.0 |
| 29.6 | 9.0 |

### EXAMPLE-1

Benzyl (2S,3aR,7aS)-1-[(2S)-2-[[(1S)-1-(Ethoxycarbonyl)-3-phenylpropyl]amino]-1-oxopropyl]octahydro-1H-indole-2-carboxylate (2 gm) obtained as per U.S. Pat. No. 4,933,361 is hydrogenated at 1 atm. pressure in ethanol (80 ml) in the presence of 10% Pd-C (200 mg) at 200 to 25° C. for 2 hours. The catalyst is filtered off and the filtrate was evaporated. Trandolapril is obtained as a foam.

### EXAMPLE-2

Trandolapril (1.5 gm) obtained by the process described by example-1 is mixed with diisopropyl ether (30 ml) and refluxed for 30 minutes. Then the solution is cooled to 20-25° C. and maintained for 1 hour and the crystals are collected by filtration and dried to give 1.0 gm of Form I crystalline polymorph of trandolapril.

### EXAMPLE-3

Trandolapril (1.5 gm) obtained by the process described in example-1 is dissolved in ethyl acetate (30 ml) and refluxed for 30 minutes. The solution thus obtained is cooled to 20-25° C. and maintained for 30 minutes and the crystals obtained are collected by filtration. The crystals are dried to obtain 1.2 gm of Form II crystalline polymorph of trandolapril.

### EXAMPLE-4

Example-2 is repeated using Form II crystalline polymorph of trandolapril instead of trandolapril obtained by the process described in example-1 to obtain 1.2 gm of Form-1 crystalline form of trandolapril.

### EXAMPLE-5

Example-3 is repeated using Form I of crystalline polymorph of trandolapril instead of trandolapril obtained by the process described in example-1 to obtain 1.1 gm of Form II of crystalline polymorph of trandolapril.

### EXAMPLE-6

Example-2 is repeated by seeding the solution during maintenance at 20-25° C. with Form I crystalline polymorph of trandolapril to give Form I crystalline polymorph of trandolapril.

### EXAMPLE-7

Example-3 is repeated by seeding the solution during maintenance at 20-25° C. with Form II crystalline polymorph of trandolapril to obtain Form II crystalline polymorph of trandolapril.

1. A crystalline polymorph of trandolapril (Form I), the x-ray powder diffractogram of which shows characteristic peaks expressed as 20 values at approximately 7.7, 9.0, 11.0, 12.6, 14.9, 15.5, 15.9, 17.0, 17.4, 17.7, 18.8, 19.9, 20.5, 23.2, 24.3, 29.0 degrees.

2. A crystalline polymorph of trandolapril (Form I) exhibiting an x-ray powder diffractogram of FIG. 1.

3. A process for the preparation of a crystalline polymorph of trandolapril (Form I) comprising the steps of:
a) mixing either i) trandolapril obtained by a previously known method or ii) a Form II crystalline polymorph of trandolapril and diisopropyl ether;
b) refluxing for about 15 to about 45 minutes; and
c) cooling to about 15° C. to about 35° C. and maintaining at about 15° C. to about 35° C. for about 15 minutes to about 2 hours, and optionally seeding with Form I crystalline polymorph of trandolapril during maintenance.

4. A process according to claim 3 wherein maintenance in step (c) is at about 20° C. to about 25° C. for about 1 hour.

5. A process according to claim 3 wherein the solution in step (c) is seeded with Form I crystalline polymorph of during maintenance.

6. A process according to claim 3 wherein trandolapril obtained by previously known method is used.

7. A process according to claim 3 wherein Form II crystalline polymorph of trandolapril is used.

8. A crystalline polymorph of trandolapril (Form II), the x-ray powder diffractogram of which shows characteristic peaks expressed as 20 values at approximately 7.3, 8.9, 12.2, 12.5, 14.6, 17.0, 17.8, 18.7, 19.8, 21.5, 22.1, 25.2, 27.8, 29.6 degrees.

9. A crystalline polymorph of trandolapril (Form II) exhibiting an x-ray powder diffractogram of FIG. 2.

10. A process for the preparation of a crystalline polymorph of trandolapril (Form II) comprising the steps of:
a) mixing either i) trandolapril obtained by a previously known method or ii) a Form I crystalline polymorph of trandolapril and ethylacetae;
b) refluxing for about 15 to about 45 minutes; and
c) cooling to about 15° C. to about 35° C. and maintaining the solution at about 15° C. to about 35° C. for about 15 minutes to about 3 hours, and optionally seeding with Form II crystalline polymorph of trandolapril during maintenance.

11. A process according to claim 10 wherein maintenance in step (c) is at about 20° C. to about 25° C. for about 30 minutes.

12. A process according to claim 10 wherein the solution in step (c) is seeded with Form II crystalline polymorph of during maintenance.

13. A process according to claim 10 wherein trandolapril obtained by previously known method is used.

14. A process according to claim 10 wherein Form I crystalline polymorph of trandolapril is used.

15. A pharmaceutical composition comprising either Form I crystalline form or Form II crystalline form or a mixture thereof of trandolapril; and a pharmaceutically acceptable carrier.

16. A pharmaceutical composition as claimed in claim 15 wherein Form I crystalline form of trandolapril is used.

17. A pharmaceutical composition as claimed in claim 15 wherein Form II crystalline form of trandolapril is used.
